(19) United States
(12) Patent Application Publication
(10) Pub. No.: US 2019/0374940 A1
(43) Pub. Date: Dec. 12, 2019

(54) LATERAL-FLOW MICROFLUIDIC CHIP AND FLOW VELOCITY CONTROL METHOD THEREOF

(71) Applicants: The Industry & Academic Cooperation in Chungnam National University (IAC), Daejeon (KR); ISIN TECHNOLOGY CO., LTD., Gwangju (KR)

(72) Inventors: Chang Soo LEE, Daejeon (KR); Seong Geun JEONG, Seosan-si (KR); Jin Hyeon LEE, Daejeon (KR); Na Mi SONG, Cheongju-si (KR); Chang Hwan YANG, Daejeon (KR)

(73) Assignees: The Industry & Academic Cooperation in Chungnam National University (IAC), Daejeon (KR); ISIN TECHNOLOGY CO., LTD., Gwangju (KR)

(21) Appl. No.: 16/227,185

(22) Filed: Dec. 20, 2018

(30) Foreign Application Priority Data
Jun. 11, 2018 (KR) 10-2018-0066917

(51) Int. Cl. B01L 3/00; G01N 33/53

(52) U.S. Cl. CPC B01L 3/50273; G01N 33/5302; B01L 2400/0403; B01L 2300/126; B01L 2200/06

(57) ABSTRACT

The present disclosure relates to a method of accelerating a flow velocity in a lateral-flow microfluidic chip in which an analysis time is not delayed while sequential reactions are possible in the lateral-flow microfluidic chip by accelerating a flow velocity in at least a section of a channel, it is easy to manufacture the microfluidic chip for applying the method, and it is possible to mass-produce the microfluidic chip, and more particularly, by increasing a vapor pressure around a specific channel, a flow velocity of a fluid in the corresponding channel is accelerated.

### CROSS-REFERENCE TO RELATED APPLICATION

This application claims priority to and the benefit of Korean Patent Application No. 10-2018-0066917, filed on Jun. 11, 2018, the disclosure of which is incorporated herein by reference in its entirety.

### BACKGROUND

### 1. Field of the Invention

The present disclosure relates to a lateral-flow microfluidic chip and a method of controlling a flow velocity of a channel in the lateral-flow microfluidic chip.

### 2. Discussion of Related Art

With the advent of aging society, the relative proportion of working age population has been decreasing, and with an increase of elderly population who have a higher incidence of diseases, medical diagnosis costs have become a serious problem. Even developing countries where medical insurance coverage is not well-established are experiencing difficulties due to costs for diagnosing diseases of individuals. In order to solve such problems, low-cost diagnostic equipment capable of diagnosing a disease with a low cost in medical institutions or households is necessary.

A lateral-flow microfluidic chip is an analytic tool using the fact that a liquid sample moves laterally due to a capillary phenomenon. For the lateral flow microfluidic chip, a material capable of flowing laterally, e.g., paper or polymers having fine structure, are used. The term paper chips in the description herein, is not limited to microfluidic chips formed of a paper material and encompasses all microfluidic chips formed of various materials capable of using the lateral flow phenomenon. Since paper chips are generally inexpensive, allow analysis with even a small amount of sample, can be discarded by incineration such, and do not require particular equipment or technology for detection during analysis. The paper chips are user-friendly, and have been widely used in point-of-care (POC) analysis.

According to a diagnostic method conventionally referred to as lateral flow test (LFT), signals appear due to binding of a primary antibody in a control band, and signals appear due to binding of a secondary antibody of a substance in a test band. When a target substance (antigen) included in a sample moves along a channel due to the capillary phenomenon, the target substance and a labeled primary antibody (e.g., antibody to which gold nanoparticles are attached) placed at a specific point primarily bind to each other and form a complex (target substance+primary antibody). Then, the complex continuously moves due to the continuous capillary phenomenon, and the complex and the secondary antibody bind to each other in the test band. On the other hand, the labeled primary antibody that does not bind to an antigen and does not form a complex, binds to a tertiary antibody placed in the control band, and shows two signal lines. When the target substance is not included in the sample, only the primary antibody binds in the control band and shows a single signal line. The test method has advantages in that detection is intuitive, simple, and takes short time, but has a disadvantage in that sensitivity of diagnosis is low.

Conversely, enzyme linked immunosorbent assay (ELISA) is a method in which a target substance is caused to react with a well to which a primary antibody is attached and then a labeled secondary antibody is caused to bind to the reacted target substance so as to perform analysis by a colorimetric reaction. Although the ELISA has an advantage over the LFT in that sensitivity is high, separate equipment is necessary for quantitative analysis and sequential reactions are required after introduction of a sample. For sequential reaction, the sample, a washing solution, the secondary antibody, and the like have to be injected into designated positions at certain time intervals for analysis to be performed using paper chips. FIG. 7 illustrates an example of a paper chip for the ELISA, in which a user has to sequentially inject a sample or solvent into positions of a primary antibody (gold-labeled detection antibody), a washing solution, and a secondary antibody (gold enhancement solution), in addition to injecting a sample for detection. However, an erroneous result may be derived when unskilled users fail to inject samples according to predetermined positions, orders, and time intervals, and even skilled users have to endure inconvenience of having to perform the task several times. Accordingly, to obtain a reproducible result while allowing convenient use by unskilled general users, controlling flow velocity of a fluid in a specific channel is essential to allow sequential reactions even with one task.

Conventional methods for controlling flow velocity of a fluid in paper chips may be mainly classified into physical methods and chemical methods.

The physical methods include methods in which a length, width, or height of a channel is adjusted to delay a flowing time, sugar is deposited on a channel to increase viscosity of a fluid flowing thereon, and a temperature of a channel is adjusted to decrease a flow velocity of a fluid. In addition, the physical methods include cases in which a magnetic valve is used, or a melting bridge manufactured by pullulan or sugar is placed between channels so that, after a fluid flows for a predetermined amount of time, the bridge melts and disappears and thus delays the fluid flow. The chemical methods include a method in which a channel is treated with a hydrophobic substance that turns hydrophilic upon contact with hydrogen peroxide so that a flow velocity of a fluid is adjusted according to a concentration of hydrogen peroxide in a sample.

Among the above methods, the method in which a flow velocity is controlled by changing the shape (length, width, depth) of a channel allows flow velocity in a specific channel to be adjusted simply by designing the shape of the channel without requiring an additional process. Accordingly, the method has an advantage in that mass-production by a roll-to-roll process is still possible. On the contrary, the method in which a channel is treated with an additional substance at every specific position or a valve is installed not only requires a separate process but also requires that the additional substance or the valve be aligned at accurate positions of the corresponding channel such that a manufacturing cost of the paper chips increases. Also, all of conventional method controlling flow velocity by decreasing way.

In an analysis using the paper chips, a time taken for obtaining a test result is one of main factors that determine practical use of a product. However, since a flow velocity adjustment in a specific channel for sequential reactions is performed by delaying the flow velocity in all of the above-described methods of the related art, the methods have a problem in that a time taken for analysis is prolonged.

### SUMMARY OF THE INVENTION

To solve the problems of the related art, it is an aspect of the present disclosure to provide a method of accelerating a flow velocity in a lateral-flow microfluidic chip capable of accelerating a flow velocity in at least a part of a channel in which a sample flows.

It is another aspect of the present disclosure to provide a lateral-flow microfluidic chip to which the flow velocity accelerating method is applied.

It is still another aspect of the present disclosure to provide a microfluidic chip for sample analysis by enzyme linked immunosorbent assay (ELISA) through sequential reactions, as a specific use example of the lateral-flow microfluidic chip.

The present disclosure for achieving the above-mentioned aspects relates to a method of adjusting a flow velocity by increasing a vapor pressure around at least a part of a channel so that a flow velocity of a fluid in the corresponding channel is accelerated, and a microfluidic chip to which the method is applied.

In the present specification and the appended claims, the expression “at least a part of a channel” refers to at least a part of a single channel as well as at least a part of at least some channels among a plurality of channels when a microfluidic chip includes the plurality of channels. For example, the expression “accelerate a flow velocity in at least a part of a channel” refers to accelerating a flow velocity in at least a part of a single channel as well as accelerating a flow velocity in at least a part of at least some channels among a plurality of channels.

The microfluidic chip according to the present disclosure uses lateral flow and may be formed of any material capable of moving a fluid by the capillary phenomenon. The most typical example of the material may include paper, and the material may also include polymers having a fine structure, but the material is not limited thereto.

Since a fluid in the lateral flow microfluidic chip flows due to the capillary phenomenon, the flow velocity is decreased due to evaporation of a fluid which occurs during the flow of the fluid. Therefore, when a vapor pressure is increased, the evaporation of the fluid is suppressed, and, as a result, the flow velocity may be accelerated in comparison to when the evaporation is not suppressed.

According to the present disclosure, the increase of the vapor pressure around a channel may be achieved by a separate vapor supply device outside the channel and also achieved by providing a liquid reservoir, which is configured to supply vapor to portions around the channel, in the microfluidic chip. Through a long period of research, the present inventor reached a conclusion that, as a liquid in the liquid reservoir naturally evaporates, the evaporation causes an increase in a vapor pressure around an adjacent channel, and as a result, causes acceleration of a flow velocity at which a sample flows in a channel, and experimentally confirmed the conclusion. It is also verified that separate vaper supply device can reduce the evaporation of the fluid, so the flow velocity of cannel can be accelerated. The present disclosure provides a lateral-flow microfluidic chip to which the flow velocity adjusting method is applied. That is, the lateral-flow microfluidic chip of the present disclosure relates to a lateral-flow microfluidic chip in which a flow velocity in a channel is adjusted and sequential reactions occur, wherein a liquid reservoir is formed adjacent to at least a part of the channel and a flow velocity in the corresponding part is adjusted. As described above, according to the present disclosure, in addition to being capable of adjusting a flow velocity in a single channel, as illustrated in FIGS. 2A-1 to 2C, flow velocity in all of the channels may be adjusted when a plurality of channels are present, and as illustrated in FIGS. 5A and 5B, and FIGS. 6A-1 to 6B, a flow velocity in at least a part of at least some channels may be adjusted when a plurality of channels are present.

As described above, since a liquid in the liquid reservoir increases a vapor pressure around a channel and suppresses evaporation of a fluid in the channel by evaporating, there is an effect in that a flow velocity is accelerated. In order to effectively increase the vapor pressure by the liquid reservoir, preferably, a width of the liquid reservoir is 1 to 10 times a width of the channel. When the width of the liquid reservoir is too narrow, the amount of liquid in the liquid reservoir is too small and is thus insufficient for exhibiting the acceleration effect. Although the effect of increasing a local vapor pressure is enhanced as the amount of liquid in the liquid reservoir is larger, when the width of the liquid reservoir is larger than 10 times the width of the channel, not only the size of the microfluidic chip is increased, but also the efficiency is lower in comparison to when the width of the liquid reservoir is smaller than 10 times the width of the channel. Of course, a degree of acceleration may be adjusted by the width of the liquid reservoir.

Even with microfluidic chips of the same design, an influence thereof on a vapor pressure of a channel varies in accordance with a type of liquid contained in the liquid reservoir. That is, even when the same amount of liquid is contained, in the case of a liquid with high volatility, an amount of evaporated liquid is greater and a vapor pressure around a channel may be more effectively increased such that the accelerating effect is greater in comparison to a liquid with low volatility. Accordingly, in the microfluidic chip of the present disclosure, a degree of acceleration may be adjusted in accordance with the type of liquid contained in the liquid reservoir.

When the liquid reservoir is formed separately from a channel at a side of the channel, since an influence of the liquid reservoir on a flow velocity is greater as a gap between the channel and the liquid reservoir is narrower, the degree of acceleration may be adjusted by adjusting the gap between the channel and the liquid reservoir. In this case, in consideration of the efficiency of spatial arrangement and the accelerating effect of the microfluidic chip, preferably, the gap between the channel and the liquid reservoir is 5 times or less the width of the channel. According to an embodiment of the present disclosure, the liquid reservoir may be formed at the side of the channel. Although the liquid reservoir may be formed only at one side of the channel, the accelerating effect is greater when the liquid reservoir is formed at both sides of the channel. Accordingly, in order to allow more efficient acceleration, it is more preferable for the liquid reservoir to be formed at both sides of the channel.

According to Example 1 of the present disclosure, when using a lateral-flow microfluidic chip that includes a separate liquid reservoir which is separated from a channel, first, a liquid has to be injected into the liquid reservoir, and then a fluid has to be injected into a sample pad. Accordingly, an injection of a fluid has to be performed at least two times. To solve such an inconvenience, as in Example 2 below, a microfluidic chip may be designed such that a channel is formed at a side of a sample pad and the sample pad serves as a liquid reservoir. According to such design, a sequential flow of a fluid is possible with one injection of a sample solution without a separate process of applying a sample to the liquid reservoir.

The lateral flow microfluidic chip of the present disclosure may be used as, for example, a chip for sample analysis by the ELISA.

The microfluidic chip according to the present disclosure may be manufactured by a suitable method according to a material and use thereof. Concisely, for example, the microfluidic chip may be manufactured by forming a channel by printing a wax pattern having a shape of a boundary of the channel on a sheet of paper and then heat-treating the wax pattern so that the wax pattern penetrates the sheet of paper. According to the above method, even without a separate process or physical operation, simply by designing a channel to have a liquid reservoir, the lateral flow-based microfluidic chip which allows sequential reactions may be manufactured conveniently and economically. Also, a means of supplying a vapor pressure is not limited according to an accelerating method of the present disclosure. Although the embodiment below relates to a case in which a liquid reservoir is provided in the microfluidic chip, the present disclosure is not limited thereto. For example, the present disclosure includes an embodiment in which vapor is supplied by an external device such as a humidifier or a humidifying chamber.

As described above, according to the method of controlling a flow velocity in a lateral-flow microfluidic chip of the present disclosure, while the advantages of the conventional microfluidic chip are maintained, a flow velocity in a corresponding part of channel may be accelerated such that a time taken for analysis using the microfluidic chip may be shortened.

The lateral-flow microfluidic chip of the present disclosure may be applied and used as a microfluidic chip for analysis using sequential reactions of a sample by designing for various uses. More specifically, the lateral-flow microfluidic chip of the present disclosure may be applied to a microfluidic chip for biochemical analysis requiring sequential reactions such as the ELISA.

According to present disclosure, it is easy to manufacture the microfluidic chip for applying the method, and it is possible to mass-produce the microfluidic chip, and the lateral-flow microfluidic chip to which the method is applied.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIGS. 1A-1 to 1C-2 illustrate design and three-dimensional schematic diagrams of a paper chip in which a liquid reservoir is formed according to an example and conventional paper chip;

FIGS. 2A-1 to 2C illustrate photographs and graphs that show a flow velocity in the paper chip in accordance with whether the liquid reservoir is formed in the example of FIGS. 1A-1 to 1C-2;

FIGS. 3A and 3B illustrate graphs that show an influence of a gap between the liquid reservoir and a channel on the flow velocity;

FIG. 4 illustrates an image that shows an influence of a vapor pressure of a liquid contained in the liquid reservoir on the flow velocity;

FIGS. 5A and 5B illustrate design of a paper chip in which a liquid reservoir is formed according to another example;

FIGS. 6A-1 to 6B illustrate photographs and graphs that show a flow velocity in the paper chip in accordance with whether the liquid reservoir is formed in the example of FIGS. 5A and 5B; and

FIG. 7 illustrates a schematic diagram of a paper chip for the ELISA of the related art.

### DETAILED DESCRIPTION OF EXEMPLARY EMBODIMENTS

The present disclosure will be described in more detail below with reference to the appended examples. However, such examples are merely examples for easily describing the technical idea and the scope of the present disclosure, and the technical scope of the present disclosure is not limited or changed by such examples. It should be apparent to those of ordinary skill in the art that various modifications and changes may be made within the scope of the technical idea of the present disclosure on the basis of such examples.

### EXAMPLES

### Example 1: Check Acceleration of Fluid in Paper Chip to which Liquid Reservoir is Adopted

1) Check Influence of Liquid Reservoir on Flow Velocity

It was predicted that a flow velocity would increase in a paper chip when a liquid reservoir is placed at both sides of a channel and a vapor pressure is selectively increased in the channel, and this was confirmed through an experiment.

For the confirmation, on a sheet of Whatman 3MM chromatography paper, a circular sample injecting portion having a diameter (inner diameter) of 12 mm and a shape of a channel which is connected to the injecting portion and has a width of 2 mm and length of 60.5 mm were designed using Adobe Illustrator CS6 program. A liquid reservoir having a width of 12.5 mm and a length of 57.2 mm was designed to be disposed at both sides of the channel at a distance of 1.5 mm from the channel. For the comparison of flow velocities, a paper chip in which the liquid reservoir is not formed was also designed. On a bottom end of an injection port, a circular support plate was printed to prevent leakage of a sample to the outside through a bottom end of the paper during injection of the sample. Also, a sample pad ring which is concentric with the injection port and has a diameter of 11.5 mm which is smaller than that of the injection port was designed to be included in the injection port of the sample. In this way, the sample was prevented from coming into direct contact with the channel during the injection of the sample.

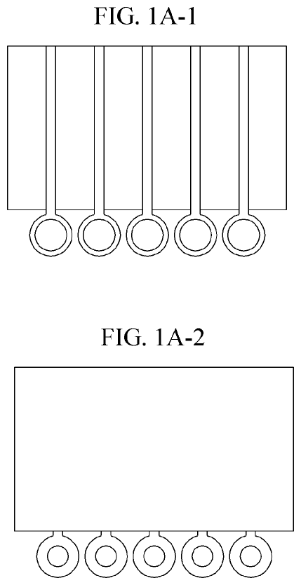

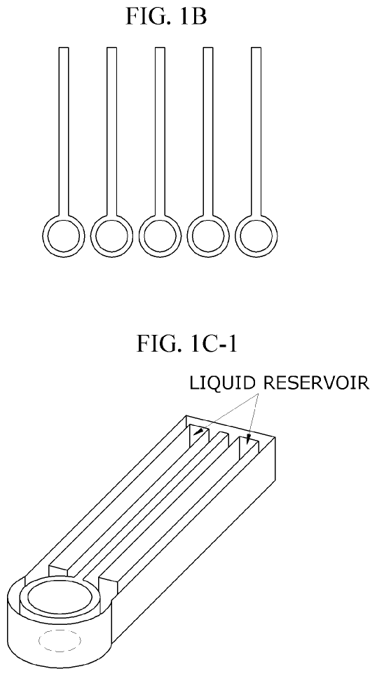

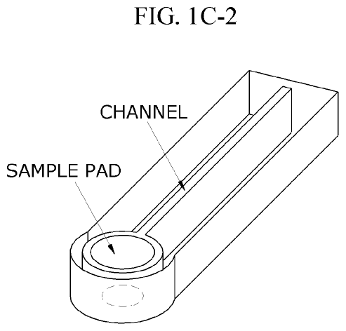

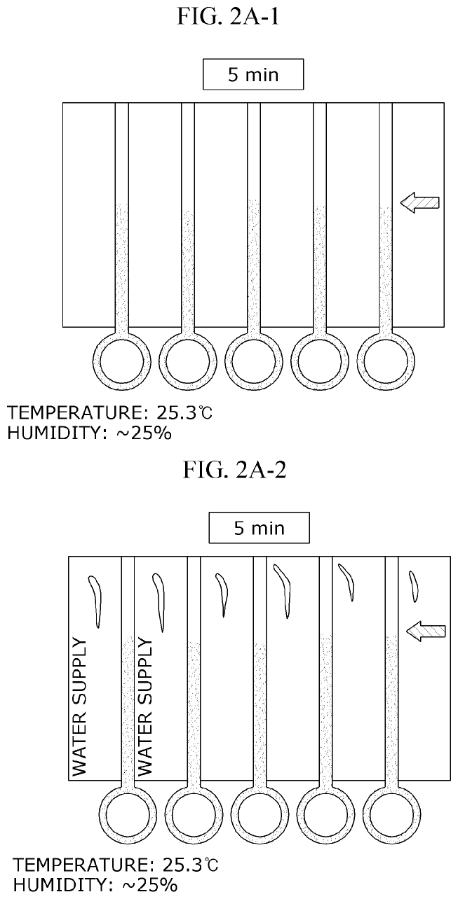

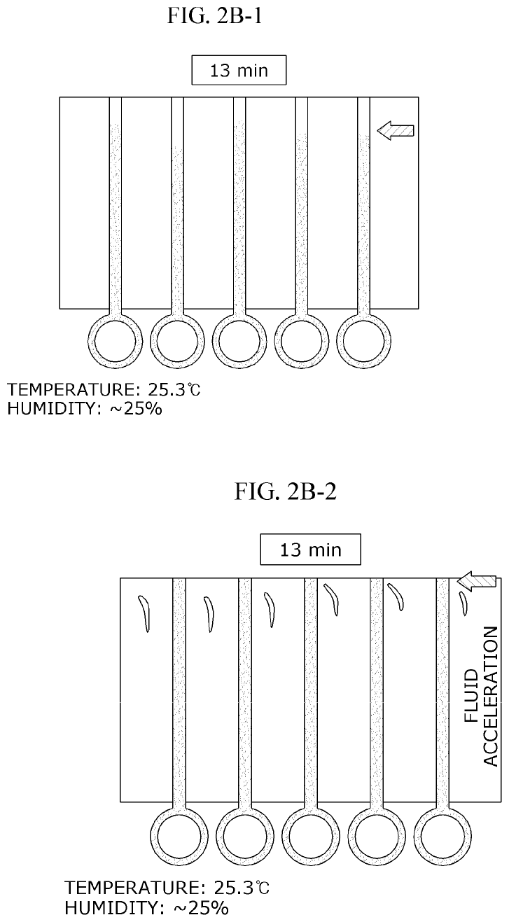

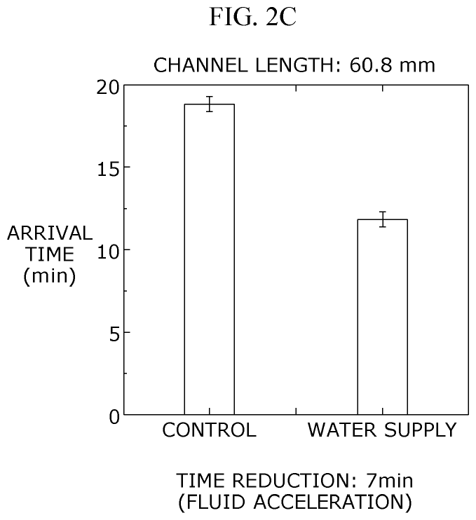

FIG. 1A-1 shows design of a front surface and FIG. 1A-2 shows a rear surface of the paper chip in which the liquid reservoir is formed according to the present example, and FIG. 1B shows design of a front surface of the conventional paper chip in which the liquid reservoir is not formed according to the related art. The patterns of the designs were printed using a wax printer (Xerox ColorQube 8570) such that the shape of the channel at the front and rear surfaces was printed with wax, and the sample pad ring was printed. Then, by heat-treating the patterns at Speed 2 at 160° C. using a laminator (PhotoLami-350R10), the wax printed on the front surface and the rear surface was caused to penetrate the sheet of paper and form a closed channel. FIG. 1C-1 illustrates three-dimensional schematic diagrams of a paper chip in which the liquid reservoir manufactured by the above method is formed. FIG. 1C-2 shows a paper chip without the liquid reservoir.

The paper chip manufactured by the above method was fixed to be horizontally arranged, and 1,500 μl of distilled water was dropped on each liquid reservoir by using a pipette. 120 μl of distilled water was simultaneously dropped on five sample pads by using a pipette, and a time at which the fluid reached an end of a channel was measured. FIGS. 2A-1 to 2B-2 is a photograph showing a flow velocity in the paper chip in accordance with whether the liquid reservoir is formed, and FIG. 2C is a graph showing a time taken for the fluid to reach the end of the channel. As seen in FIG. 2C, while the fluid reached the end of the channel in an average of 18 minutes 49 seconds in the paper chip of the related art to which the liquid reservoir is not adopted, the fluid reached the end of the channel in 12 minutes 8 seconds in the paper chip to which the liquid reservoir is adopted. That is, it was confirmed that, due to adoption of the liquid reservoir, the fluid reached the end of the channel 6 minutes 41 seconds faster, and the flow velocity increased by about 1.55 times. This is considered to be due to a decrease in an evaporation rate of a fluid in a channel which is due to an increase in humidity level of a section of the paper chip caused by the liquid reservoir.

2) Check Influence of Gap Between Liquid Reservoir and Channel on Flow Velocity

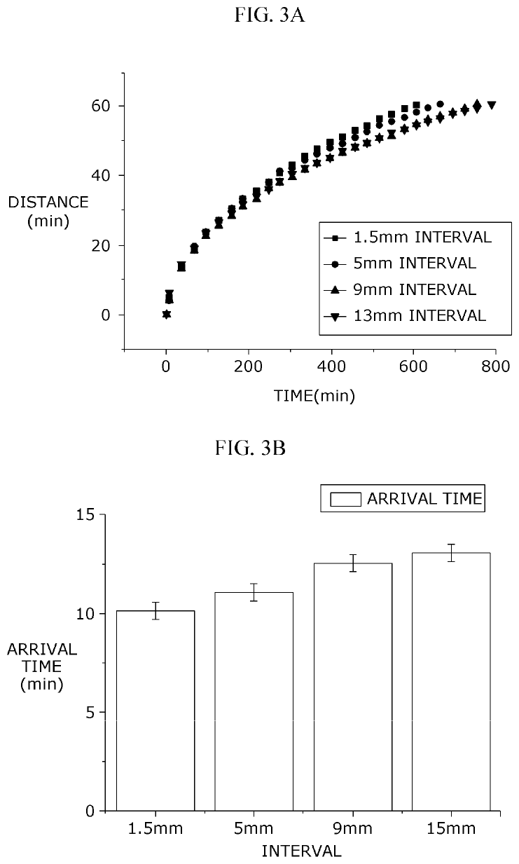

To check the influence of the gap between the liquid reservoir and the channel on the flow velocity, a flow time was measured by the same method as in 1) with respect to the gaps were changed, from the design of the channel in 1), to be 1.5 mm, 5 mm, 9 mm, and 13 mm. FIG. 3A is a graph showing a travel distance of a fluid with time and a time taken for the fluid to reach the end of the channel in each channel. And it can be seen from FIG. 3B that the flow velocity of the fluid is higher as the gap between the liquid reservoir and the channel is narrower. In the channels which are distant from the liquid reservoir at 1.5 mm, 5 mm, 9 mm, and 13 mm, the times taken for the fluid to reach the end of the channel were 606 seconds, 665 seconds, 755 seconds, and 788 seconds, respectively.

3) Check Influence of Type of Liquid Contained in Liquid Reservoir on Flow Velocity

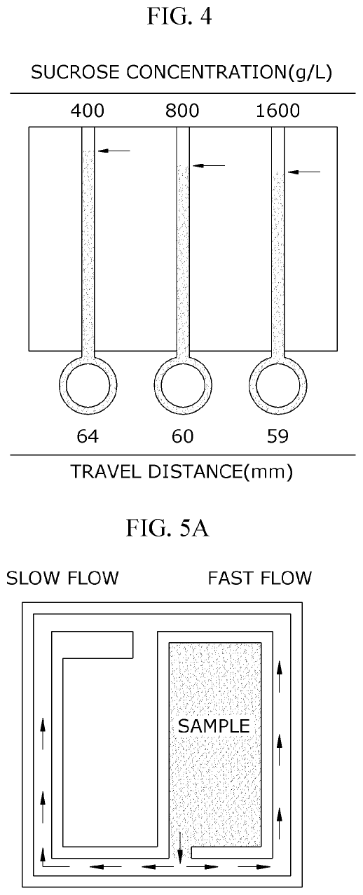

To check the influence of the type of liquid contained in the liquid reservoir on the flow velocity, sugar water having different concentrations was poured into the liquid reservoir of the paper chip manufactured in 1), and a flow velocity of a fluid was measured. Except for injecting sugar water having concentrations of 400, 800, and 1600 g/L instead of water in the liquid reservoir, the flow velocity was measured by the same method as in 1). FIG. 4 is an image showing a travel distance 25 minutes after sample injection, and it can be seen from FIG. 4 that the travel distance of the fluid is shorter during the same time as the concentration of sugar is higher. Since the boiling point increases and the vapor pressure decreases as the concentration of sugar is higher, the above result indicates that a degree of acceleration may be adjusted in accordance with a vapor pressure property of a liquid contained in the liquid reservoir.

### Example 2: Check Acceleration of Fluid in Paper Chip in which Sample Pad is Utilized as Liquid Reservoir

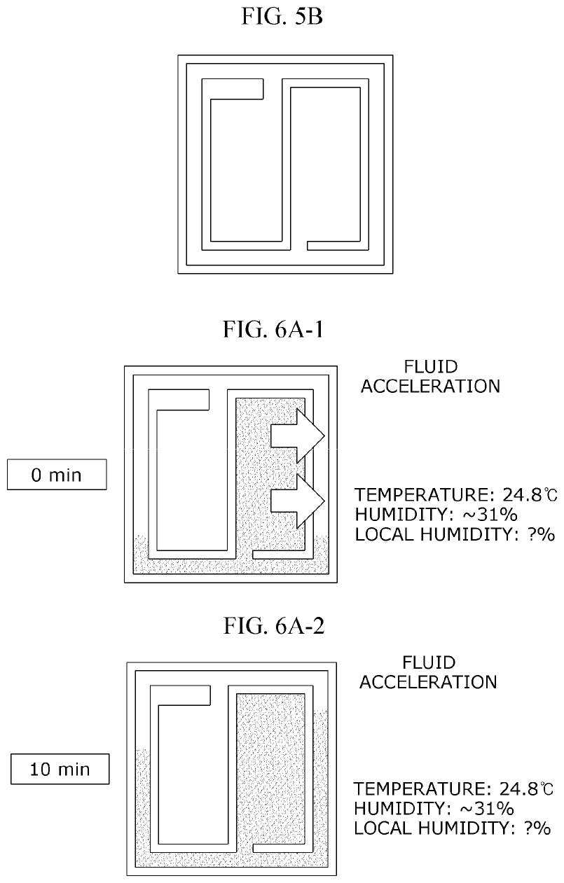

To eliminate an inconvenience of having to separately pour a liquid into a separately-formed liquid reservoir as in Example 1, usefulness of design of a channel of a paper chip in which a sample pad itself may be used as a liquid reservoir was confirmed. FIG. 5A is a conceptual diagram of a channel of the paper chip in which a sample pad serves as a liquid reservoir, and FIG. 5B show designs of a front surface of a sheet of paper using Adobe Illustrator CS6 program. The method of manufacturing the paper chip is the same as that in Example 1, and numerical values used in the design are indicated in Table 1.

TABLE 1

| Element | Size |
|---|---|
| Channel width | 2 mm |
| Gap | 1 mm |
| Sample pad | 13 mm*26 mm |

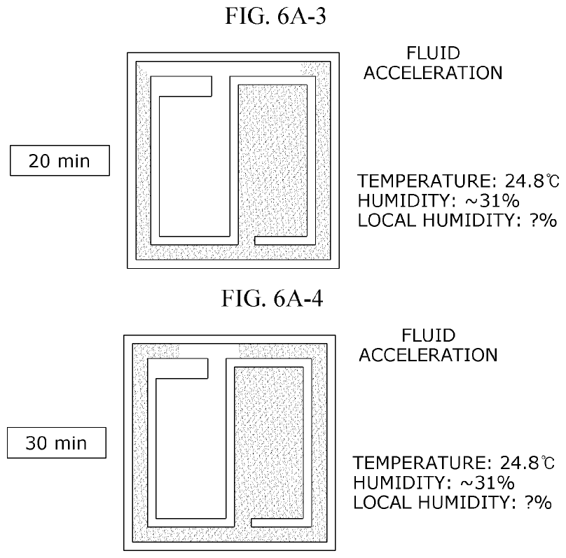

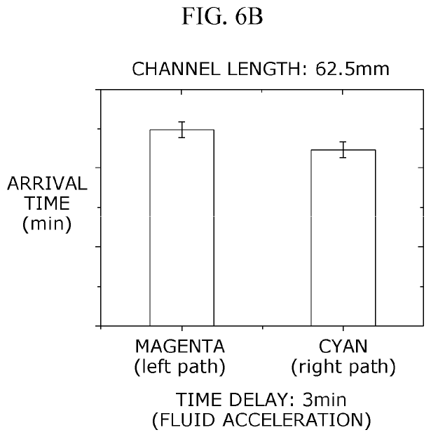

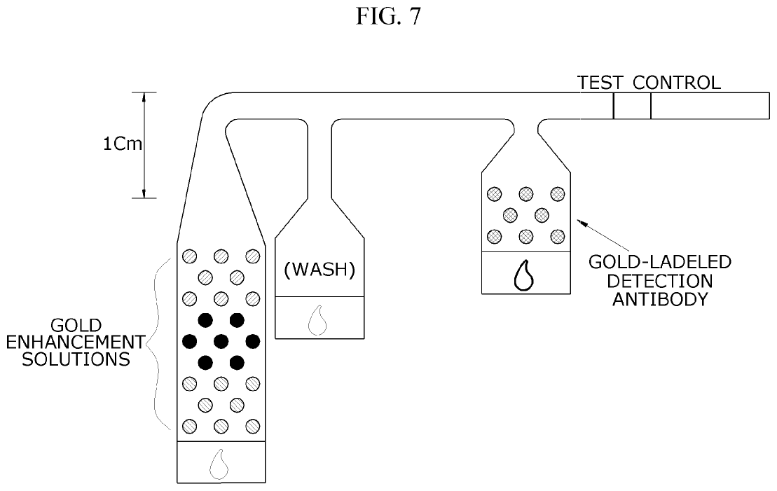

To facilitate observation of the flow of the fluid in the completed paper chip, 2 μl of magenta ink was adsorbed onto a corner of left path, 2 μl of cyan ink was adsorbed onto a corner of right path, and then the adsorbed ink was dried. The paper chip in which the ink was dried was fixed to be horizontally arranged, 800 pi of distilled water was dropped on each sample pad by using a pipette, and the flow of the fluid was observed. FIGS. 6A-1 to 6A-4 are photographs of the paper chip with time, and FIG. 6B is a graph showing time taken for the fluid to reach the end of each channel. As it can be seen in FIGS. 6A-1 to 6B, an average time taken for the fluid to reach the end of the channel was 22 minutes 17 seconds along the right path and 25 minutes along the left path, and thus was about 2 minutes 43 seconds faster along the right path. This indicates that the sample pad may simultaneously serve as the liquid reservoir and accelerate the flow of the fluid in an adjacent channel. As indicated by Example 2 above, in the present disclosure, the sample pad may be used as the liquid reservoir and may selectively accelerate a flow velocity in some or all of a plurality of channels.

Meanwhile, the shape of the liquid reservoir may be deformed in various ways to adjust the flow velocity. Although an example in which the liquid reservoir has a rectangular shape that abuts a wall of the channel has been described above with reference to the above examples, the shape of the liquid reservoir is not limited thereto, and the liquid reservoir may have a circular, triangular, square, pentagonal, or any arbitrary shape.

Since a portion of the liquid reservoir which is the closest to the channel has the greatest influence on acceleration of a fluid, the flow velocity in the channel may be adjusted by adjusting a region in the vicinity of the channel while deforming the shape of the liquid reservoir.

And the total capacity of the liquid reservoir determines the acceleration time, as well as the amount of liquid in the liquid reservoir.

The present disclosure has been described above with reference to a few examples, but it should be apparent to those of ordinary skill in the art that various modifications and changes are possible within the scope of the technical idea of the present disclosure. The scope of the present disclosure is not limited by the above description and examples, and is defined by the claims below.

What is claimed is:

1. A method of controlling a flow velocity in a lateral-flow microfluidic chip, the method comprising:
increasing a vapor pressure around at least a part of a channel.

2. The method of claim 1, wherein the increasing of the vapor pressure includes:
forming a liquid reservoir around at least a portion of the channel; and
filling the liquid reservoir with a liquid.

3. The method of claim 2, wherein the increasing of the vapor pressure further includes adjusting the increased vapor pressure by changing a concentration of the liquid with which the liquid reservoir is filled.

4. The method of claim 2, wherein the increasing of the vapor pressure further includes adjusting the increased vapor pressure by deforming a shape of the liquid reservoir.

5. A lateral-flow microfluidic chip, comprising:
a channel in which a sample flows; and
a liquid reservoir formed at a side of at least a part of the channel.

6. The lateral-flow microfluidic chip of claim 5, wherein the liquid reservoir is separated from the channel.

7. The lateral-flow microfluidic chip of claim 5, wherein the liquid reservoir is a sample pad.

8. The lateral-flow microfluidic chip of claim 5, wherein a degree of acceleration is adjusted by a gap between the channel and the liquid reservoir.

9. The lateral-flow microfluidic chip of claim 5, wherein a degree of acceleration is adjusted by a width of the liquid reservoir.

10. The lateral-flow microfluidic chip of claim 5, wherein a degree of acceleration is adjusted by a type of liquid contained in the liquid reservoir.

11. The lateral-flow microfluidic chip of claim 7, wherein an acceleration time is adjusted in accordance with a capacity of the liquid reservoir.

12. The lateral-flow microfluidic chip of claim 5, wherein the liquid reservoir is formed at both sides of the corresponding channel.

13. The lateral-flow microfluidic chip of claim 7, wherein the microfluidic chip is a chip for sample analysis by enzyme linked immunosorbent assay (ELISA).

14. The lateral-flow microfluidic chip of claim 7, wherein the microfluidic chip is a paper chip in which a channel is formed by printing a wax pattern having a shape of a boundary of the channel on a sheet of paper and then heat-treating the wax pattern.

15. The lateral-flow microfluidic chip of claim 5, wherein the liquid reservoir is formed at a side of at least some of a plurality of channels.
